(19) United States
(12) Patent Application Publication
(10) Pub. No.: US 2018/0152354 A1
(43) Pub. Date: May 31, 2018

(54) System and Method for Transportable Data Transformation Topologies

(71) Applicant: Under Armour, Inc., Baltimore, MD (US)

(72) Inventors: Allan Glen, Denver, CO (US); Ben Hamill, Austin, TX (US); Brett Nekolny, Denver, CO (US); James Crasta, Denver, CO (US); Brad Fults, Austin, TX (US); Eric Roberts, Austin, TX (US); Chris Peters, Austin, TX (US); Andrew Harrison, Austin, TX (US)

(21) Appl. No.: 15/826,087

(22) Filed: Nov. 29, 2017

Related U.S. Application Data
(60) Provisional application No. 62/427,352, filed on Nov. 29, 2016.

(51) Int. Cl. H04L 12/24; H04L 29/08

(52) U.S. Cl. CPC H04L 41/12; H04B 1/385; H04L 67/10

(57) ABSTRACT

System and method for enabling data transformation topologies to be transported and/or distributed within a network of devices. In one embodiment, the system comprises: a network apparatus which runs a computer program configured to: (i) generate a plurality mathematical processes to be applied; and (ii) create an instruction set comprising an ordered list of ones of the processes, and information identifying one or more data types to be utilized with each of the processes. The system further comprises a plurality of user devices which run a second computer program configured to: (i) receive access to the processes and the instruction set; and (iv) perform the mathematical processes in the order provided by the instruction set to arrive at transformed data.

### RELATED APPLICATIONS

The present disclosure claims priority to co-owned, co-pending U.S. Provisional Patent Application Ser. No. 61/427,352; which is incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### FIELD

This disclosure relates generally to the field of enabling data transformation topologies to be transported and/or distributed within a network of devices. More particularly, the present disclosure relates to systems, computer programs, devices, and methods for creating a library of application components which are accessible to a plurality of user devices, and subsequently defining a sequence of performance of ones of the components to complete a transformation process on collected data.

### BACKGROUND

In recent years, health monitoring devices that are used or worn by users to measure or track physical and physiological information relating to the health and activity levels of the users have gained great popularity. Such health monitoring devices collect health data relating to a user, and enable the data to be stored, processed, and displayed to the user. In one embodiment, the health monitoring devices comprise computing devices, such as smartphones or personal computers, which are configured to execute one or more software programs for analysis of the data. Common health data analysis systems provide displays of historical health information for the user. Additionally, analysis systems may provide information relating to the user's health goals, diet advice or analysis, and exercise advice or analysis, etc. based on the collected health data. Additionally, some users have more than one health monitoring device. For example, a user may carry a smartphone throughout the day that acts as a pedometer to record steps or general activity levels: additionally the user may wear a wristband with a larger array of sensors and/or different types of sensors during workouts and/or use sensor enabled equipment.

Given the rapid advances in the field of health monitoring, many users upgrade to newer health monitoring devices over comparatively short time periods. Existing analysis systems, however, have difficulties in processing health data from multiple devices for a single user. Combining data from different hardware devices often produces inaccurate analytical results or the analysis system must discard historical health data to process only health data from a new device. Moreover, in certain instances it is advantageous to provide updates to the processing steps which are performed on the collected data. Consequently, improvements to analysis systems to improve analysis of health data for a user from multiple health monitoring devices are needed.

### SUMMARY

The present disclosure addresses the foregoing needs by disclosing, inter alia, methods, devices, systems, and computer programs for enabling data transformation topologies to be transported and/or distributed within a network of devices by creating a library of application components which are accessible to a plurality of devices, and subsequently defining sequence of performance of ones of the components to complete a transformation process on collected data.

In one aspect of the disclosure, a non-transitory, computer readable medium is disclosed. In one embodiment, the computer readable medium comprises a plurality of instructions which are configured to, when executed, cause a user device to: (i) receive access to a library comprising a plurality of processing functions; (ii) receive a transformation process instruction, the instruction comprising individual ones of the plurality of processing functions to be executed and a pre-defined order in which the individual ones of the plurality of processing functions are to be executed; (iii) obtain a plurality of data; (iv) identify at least a portion of the data for use in the transformation process; (v) access the individual ones of the plurality of processing functions identified in the instruction; (vi) execute the individual ones of the plurality of processing functions on a portion of the data in the pre-defined order to arrive at transformed data; and (vii) provide the transformed data to a user or store the data for later use.

In another aspect of the disclosure, a network apparatus configured to enable transformation topologies to be distributable to a plurality of user devices is provided. In one embodiment, the apparatus comprises: one or more interfaces; a storage apparatus; and a processor configured to execute at least one computer application thereon. In one variant, the computer application comprises a plurality of instructions which are configured to, when executed, cause the network apparatus to: (i) generate a plurality of components, each of the plurality of components comprising at least one mathematical process to be applied; (ii) transmit the plurality of components to at least one storage apparatus associated with at least one of the plurality of user devices; (iii) create an instruction set, the instruction set comprising an ordered list of multiple ones of the plurality of components, and information configured to identify one or more specific data types to be utilized with each of the multiple ones of the plurality of components; and (iv) provide the instruction set to the at least one of the plurality of user devices for execution thereby.

In yet another aspect of the disclosure, a system configured to enable implementation of data transformation topologies across a network of devices is provided. In one embodiment, the system comprises: at least one network apparatus and a plurality of user devices. In one variant, the at least one network apparatus comprising at least one first computer program, the first computer program comprising a plurality of instructions which are configured to, when executed, cause the network apparatus to: (i) generate a plurality of components, each of the plurality of components comprising at least one mathematical process to be applied; and (ii) create an instruction set, the instruction set comprising an ordered list of multiple ones of the plurality of components, and information configured to identify one or more specific data types to be utilized with each of the multiple ones of the plurality of components. In another variant, the plurality of user devices each comprises at least one second computer program, the second computer program comprising a plurality of instructions which are configured to, when executed, cause the user devices to: (i) receive access to the plurality of components; (ii) receive the instruction set; (iii) obtain the identified one or more of the specific data types; (iv) perform the multiple ones of the plurality of components in the order provided by the instruction set to arrive at transformed data; and (v) provide the transformed data to a user or store the data for later use.

These and other aspects of the disclosure shall become apparent when considered in light of the disclosure provided herein.

### BRIEF DESCRIPTION OF THE DRAWINGS

FIG. 1 is a block diagram illustrating an exemplary system for enabling data transformation topologies to be transported and/or distributed within a network of devices in accordance with one embodiment of the present disclosure.

FIG. 2 is a logical flow diagram illustrating an exemplary method for enabling data transformation topologies to be transported and/or distributed within a network of devices in accordance with one embodiment of the present disclosure.

FIG. 3 is a logical flow diagram illustrating another exemplary method for enabling data transformation topologies to be transported and/or distributed within a network of devices in accordance with one embodiment of the present disclosure.

FIG. 4 is a block diagram illustrating an exemplary client device in accordance with one embodiment of the present disclosure.

FIG. 5 is a block diagram illustrating an exemplary server apparatus in accordance with one embodiment of the present disclosure.

All Figures © Under Armour, Inc. 2016. All rights reserved.

### DETAILED DESCRIPTION

Disclosed embodiments include systems, apparatus, methods and storage media which enable data transformation topologies to be transported and/or distributed within a network of devices by creating a library of application components which are accessible to a plurality of user devices, and subsequently defining sequence of performance of ones of the components to complete a transformation process on specific ones of the collected data.

In the following detailed description, reference is made to the accompanying drawings which form a part hereof wherein like numerals designate like parts throughout, and in which is shown, by way of illustration, embodiments that may be practiced. It is to be understood that other embodiments may be utilized, and structural or logical changes may be made without departing from the scope of the present disclosure. Therefore, the following detailed description is not to be taken in a limiting sense, and the scope of embodiments is defined by the appended claims and their equivalents.

Aspects of the disclosure are disclosed in the accompanying description. Alternate embodiments of the present disclosure and their equivalents may be devised without parting from the spirit or scope of the present disclosure. It should be noted that any discussion herein regarding “one embodiment”, “an embodiment”, “an exemplary embodiment”, and the like indicate that the embodiment described may include a particular feature, structure, or characteristic, and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C). Similar logic applies to the use of the term “or” herein; i.e., “A or B” means (A), (B), or (A and B).

The terms “comprising,” “including,” “having,” and the like, as used with respect to embodiments of the present disclosure, are synonymous.

### Network Architecture

There exists a persistent need to provide flexible processes which are executed at a user device (or other transformation apparatus), without overburdening the computing system of the user device. It is further advantageous to provide updates to the processes with as little download requirement as possible. In order to do so, the present disclosure provides a system for generating and transmitting components of a process as well as instructions for their execution.

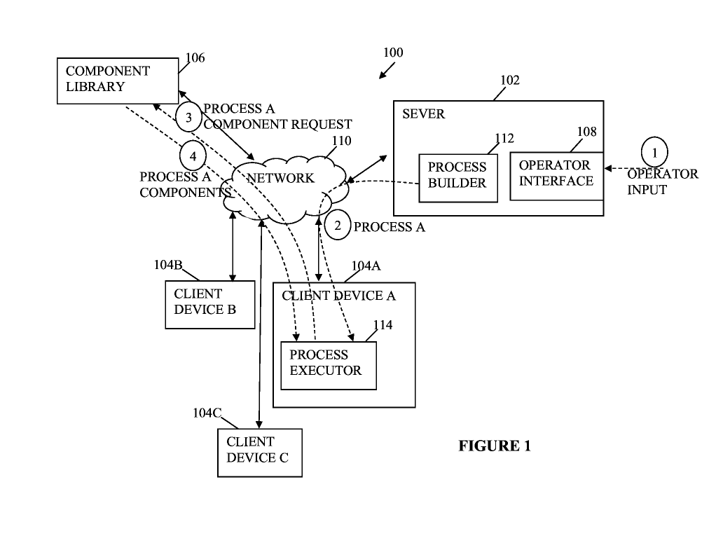

Referring now to FIG. 1, an exemplary system enabling data transformation topologies to be transported and/or distributed within a network of devices is shown. As illustrated, the system generally comprises a server apparatus 102 in communication with one or more user devices 104A, 104B, 104C, and a component library 106 via a network 110. It is noted that in FIG. 1, the solid lines represent actual connections (whether wired or wireless), and the dotted lines represent logical connections.

The network 110 which enables communication between the server 102, the plurality of user devices 104, and the component library 106 (each discussed in turn below) may comprise one or more wired and/or wireless, private and/or public network, including but not limited to, e.g., the Internet. The network 110 is, for example, a wireless local area network (WLAN), wireless wide area network (WWAN), wired network, or any other suitable communication channel. Accordingly, each of the user devices 104, server(s) 102, and component library 106 are configured with appropriate networking communication interfaces. An example of wired communication interface may include, but is not limited to, Ethernet; while examples of wireless communication interfaces may include, but are not limited to, near field communication (NFC), Bluetooth, WiFi, 4G or 5G LTE. It is further appreciated that various gateways, routers, switches, base stations, and so forth may be involved in facilitating and forwarding communication between the foregoing devices. Additionally, it is noted that the foregoing network may comprise several networks, such that the described components are distributed in various ones thereof. In alternative embodiments, the network may comprise a series of devices communicating within software via software API's.

The user devices 104, in one exemplary implementation, comprise one or more portable computerized devices which are configured to measure, obtain, monitor, generate, collect, sense, or otherwise receive biometric, environmental, activity and/or health parameters. User devices 104 may also be referred to herein as health and/or activity monitoring devices. In one variant, certain ones of the user devices 104 comprise wearable health-related parameter measurement and computing devices, such as e.g., a smart watch, an activity tracker, a heart rate monitor, a sleep tracking device, a nutrition tracking device, a foot pod or other sensor placed in an article of clothing, a smart scale, and/or smart eyeglasses. In addition, an exemplary user device 104 may comprise a smartphone having one or more of the foregoing capabilities and/or which enables user entry of the foregoing health data. Alternatively, the user device 104 is in communication with a health and/or activity monitoring device to receive data therefrom.

The sensed health parameter data comprises data which the particular device 104 is configured to collect (such as activity, biometric, and environmental data). For example, an activity tracking device is configured to collect activity data such as steps taken, distance travelled, rate or pace of a run, and/or flights of stairs climbed, etc.; a heart rate monitor is configured to collect heartbeat data; a sleep tracking device collects data relating to how much time a user/wearer spends sleeping; a nutrition tracking device collects data relating to food and drinks consumed by a user; a smart scale collects data relating to a body weight, body fat percentage, and/or body mass index (BMI), etc. Furthermore, a smartwatch and/or smartphone, may be utilized as an activity tracking device, a heart rate monitor, a sleep tracking device, and/or a nutrition tracking device. The user device 104 may comprise any of the foregoing types of devices and/or may receive collected data from a first device at one or more applications running on the user device 104.

In certain embodiments, a single user may be associated to multiple user devices 104. For example, a particular user may employ a wrist worn device for capturing activity data, a smart scale for measuring body weight and body fat percentage, a smartphone application for entering nutrition data, and a chest strap for capturing heart rate. Each device is given a unique device identifier (“device ID”); similarly, each user is given a unique user identifier (“user ID”). In this manner, as health parameter data that is obtained, generated, collected, sensed, or otherwise received at each user device 104 and relating to a particular user, it is appropriately distinguished or identified.

The exemplary user device may be further configured as a transformation apparatus, i.e., configured to transform the collected data. Such transformation may include e.g., processing data for display or human readability, deriving a metric from the data, etc. In such instances, the exemplary user device 104 may run one or more applications configured to process (e.g., transform) the collected data. Exemplary applications include e.g., UA Record™, MapMyFitness®, MyFitnessPal®, Endomondo®, etc. each owned by assignee hereof. Other health activity related monitoring applications may additionally be utilized in connection with the present disclosure, such as those specifically designed to receive information from a particular type of health monitoring device (i.e., an application which is published by the device manufacturer); the foregoing being merely representative of the general concepts of the present disclosure.

As will be discussed in greater detail below, in one exemplary embodiment the application(s) running at the user device 104 which are configured to process data utilize at least one process executor application 114. The process executor 114 follows instructions received from the server 102 (such as from a process builder application 112) in order to execute a transformation process on the collected, sensed, or otherwise obtained data. The executor 114 is configured to receive instructions, review the instructions, access a library of components, call and execute specific ones of the components in an order specified by the instructions and on data specified by the instructions. The resultant transformed data is the displayed to the user.

The server 102 as illustrated in FIG. 1 comprises one or more computerized devices operable to enable an operator to build one or more components which are utilized to build one or more processes, the processes are then provided to the user devices 104 for execution thereat. To this end, as shown, the exemplary server 102 comprises at least a process builder 112 and an operator interface 108. Additional features and components of the server 102 will be discussed in further detail below.

The action or process (also referred to as: transformation, operation, and/or procedure) refers to any process that receives the health parameter data and generates a modified or “transformed” set of health parameter data therefrom. Using a so-called “data path” associated to the data (as discussed elsewhere herein), it is advantageously possible to identify specific data on which to perform a process as well as to trace an output of transformed data (including through the multiple transformations which have been applied thereto). The processes are performed by one or more transformation apparatus. In one exemplary embodiment, one or more user devices 104 may operate as a transformation apparatus in that the user device 104 may be configured to run one or more applications thereon which transform the health parameter data (as noted above). For example, an application running on a user's smartphone (i.e., user device 104) may be configured to transform GPS location data into distance, and/or may be configured to turn elevation data into slope. Other transformation apparatus may include non-user device entities which implement one or more transformation processes (not shown).

As noted above, each step in a transformation process may be referred to herein as a “component”. In one example, a transformation step or component may comprise calculating a mathematical average, or other mathematical process. In a further embodiment, each component or transformation step is associated to an identifier (such as a uniform resource identifier (URI) or other unique identifier) which enables the particular component to be found via a search of the component library 106. According to this embodiment, the process builder 112 may generate a registry listing the specific component identifiers as the components are built/entered into the library 106, each identifier mapping to a particular mathematical formula to be executed as a transformation step. In one variant, the component registry comprises a MySQL® table having a single column that is used as a so-called whitelist. In another embodiment, alternative means for uniquely identifying each component may be used, such as UUID, etc.; the foregoing example is merely demonstrative of the overall concept.

The system 100 functions to enable an operator to input via the operator interface 108 one or more instructions to create a process within the process builder application 112, represented in FIG. 1 at item 1. The inputs may comprise creation of individual components of a process, selection of one or more components, and placement of the components into instructions which set forth the order which each is to be performed. The operator may further designate in the instructions the specific data which is to be used in each component, such as by identifying a source of data, data type, and/or data path.

In one variant, the designation of particular data may be accomplished by identifying data via a data path thereof. One system for providing a data path is discussed in co-owned, co-pending U.S. Provisional Patent No. 62/403,882 filed on Oct. 4, 2016 and entitled “System and Method for Data Tracking Within a Network of Devices”, which is now U.S. patent application Ser. No. ______ and which is incorporated herein by reference in its entirety. As discussed therein, health data from one or more health monitoring devices associated with a user is tracked or traced throughout a series of processing steps irrespective of a device which performs the processing step. In one embodiment, the tracking or tracing is accomplished by generating a unique metadata file as the data is collected and processed. The identifying metadata identifies a device which created the data. Thereafter, additional metadata is added relating to each data processing step performed and the device which performed the step. Appropriate data may be identified via the metadata file associated therewith.

Using the herein discussed methods and apparatus, individual processes may be derived which are optimized to a particular device and/or type of device. For example, processes may be derived which are optimized for e.g., the iOS operating system. Other processes may be specifically derived based on user inputs; i.e., a user may indicate that he would prefer processes which relies on a first mathematical assumption.

Once a process is built, the process builder 112 provides the completed process to the user devices 104. In the illustrated embodiment, a process, Process A, is specifically built for a first type of devices, client device 104A. Accordingly, as illustrated at item 2, Process A is provided to the process executor 114 of the client device 104A. Other, or similar processes may be provided to the remaining devices.

As will be discussed in greater detail below, the process executor 114 reviews Process A to identify the components that are required. A request is then sent from the process executor 114 to a component library 106 (item 3).

The component library 106 comprises a database or store of components uploaded thereto by the process builder application 112. The components themselves may comprise mathematical functions which are to be performed on collected data. For example, a particular component may comprise a mathematical function to transform GPS data relating to a start and end point of a workout into a value for a distance travelled. Another component may transform data relating to a start and end time as well as the distance travelled data (determined previously) by applying a different mathematical equation to arrive at an average speed. Other transformations and/or mathematical formulas or equations may be considered components consistent with the present disclosure; the foregoing being merely illustrative of the general concepts provided.

It is appreciated that in the illustrated embodiment, the component library 106 comprises a separate entity in communication with server 102 and user device(s) 104. However, in other variants, the component library 106 may be provided in part or in whole to the user device 104 for storage thereat. For example, components which are specific to a particular type of device and/or particular health monitoring applications are provided only to those devices 104 as needed. Additionally, or in the alternative, the component library 106 may be stored at the server 102 and portions thereof may be made accessible to particular devices 104. Any combination of the foregoing configurations may be utilized with equal success.

Referring back to FIG. 1, in response to the request for the components of Process A, the component library 106 returns the appropriate components (at item 4) to the user device 104. In one embodiment, the components themselves are provided. In another variant, access to the components is provided, such that the mathematical formula represented by the component is performed at e.g., the component library 106, or other processing entity. The provided components may be stored at the user device 104 temporarily in one embodiment.

In this manner, changes to individual components and/or additional or new components may be updated to the component library 106 without requiring the process execution applications running at the user device 104 to be updated entirely. That is, an existing version of a health monitoring application may continue to load and run without change because updates to the functioning of a given process of the application are made at the component library 106. In addition, new topologies or processes may be implemented at the device 104 via instructions to process different ones of the given components and/or in a different order, thereby the number and type of available transformation processes is greatly expanded without an expansion to the device requirements.

Exemplary methods of enabling data transformation topologies to be transported and/or distributed within a network of devices are discussed in further detail below.

### Methodology

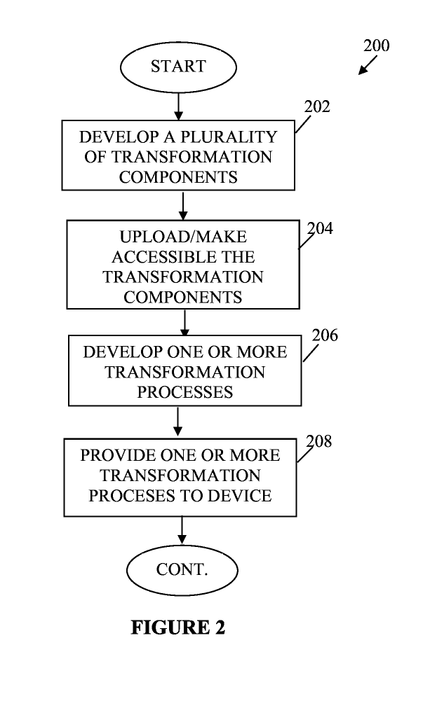

Referring now to FIG. 2, an exemplary method 200 for enabling data transformation topologies to be transported and/or distributed within a network of devices is given. As shown, per step 202, a plurality of transformation components are generated or obtained. In one embodiment, the components are generated at a process builder application 112 via user (or operator) inputs into an operator interface 108 at a server device 102. In one variant, the steps of a particular transformation process (i.e., components) are manually entered as noted above; alternatively, one or more components may be uploaded or provided from another source (not shown).

The transformation process components may identify a type and source of data to be used therein. The components may further identify a type of device (or devices) for which the component is optimized. The aforementioned identifications may be made via metadata tags associated to each component. The components themselves are each assigned a unique identifier or unique location within a component library, which may be used to call the component (discussed elsewhere herein).

Next, at step 204, server 102 causes the transformation components to be made accessible and/or to be uploaded to the user devices 104. In one embodiment, the components are uploaded for storage at a storage device associated with the user devices 104 (i.e., a component library provided at a storage device internal to the user devices 104). In another embodiment, one or more external storage device(s) download the transformation components (such as the component library 106 of FIG. 1), the storage devices are configured for communication to more than one user device 104, such that all of the devices are provided with at least one storage device from which they may access the transformation components.

As each new transformation component is created, it is provided from the server 102 to the individual user devices 104 and/or to the external component libraries 106. Additionally, as changes are made to existing components, these changes are populated to the component storage entities (e.g., at the user device 104 and/or at the external component libraries 106). For example, a particular component may exist as a first version, then, as new data sources are provided and/or mathematical changes are implemented, a newer version of the component may be derived. In this example, the new component may identify the new data source for one or more of the data fed into the component and/or may include the change to a mathematical formula performed by the component. According, as new versions of the components (or functions performed thereby) are generated, the library 106 is updated. It is further noted that in at least one embodiment, the functions are not instantly-distributable, thus, in some instances certain devices may have the newest versions, whereas others might not. As discussed herein, the ordering and arrangement of the functions or components is transportable, not necessarily the functions themselves.

At step 206, an operator at the server 102 develops one or more transformation processes using the previously provided components. That is, an operator may via the operator interface 108 to the process builder 112, build a transformation process having multiple steps (or components). The process builder 112 enables the operator to select one or more components to be executed and an order thereof. Additionally, the transformation process may specify any number of particular features of the process, for example, the operator may specify the particular data to be utilized in the process, the health monitoring device from which data should be collected, and/or may provide a hierarchy or ordered list of acceptable data and/or data sources to be used in the process.

The specific features and components of a particular transformation process are collected as “process instructions”, i.e., a set of executable steps which indicate a unique location or identity of the transformation components to be utilized, listed in order thereof as well as an identifier or tag associated with data which is to be utilized in each transformation component. It is noted that in addition to an order of the components, the process instructions may further identify branching and/or merging of streams of data. That is, in certain functions data is input, transformed, and the resultant output data is provided to more than one second component or function, this is outlined in the process instructions. In another example, the process instructions are able to identify two or more data streams which are each input to a particular function and a single data output is created.

Once a transformation process is developed, it is provided from the server 102 to the individual user devices 104 at step 208. Certain processes may be provided to particular user devices 104 based on e.g., the type of device for which the process is optimized, the particular process executor running thereat 114, the one or more health monitoring applications run thereon, etc. In another embodiment, the processes may be provided in an external library such as that provided for the components 106; such library may be made accessible to the user devices 104.

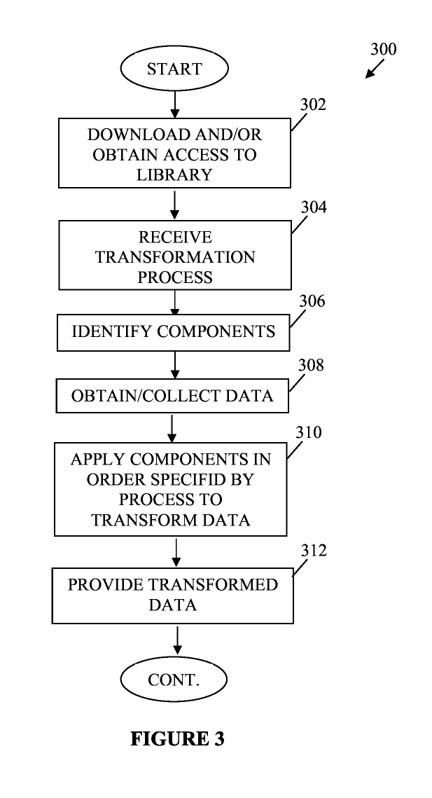

Referring now to FIG. 3, a logical flow diagram illustrating another exemplary method 300 for enabling data transformation topologies to be transported and/or distributed within a network of devices is provided. As shown, per step 302, a user device downloads a plurality of available components and/or receives access to a component library 106. As noted above, the components comprise those transformation components created by a network operator at a server device 102. Individual ones of the components may be provided to the user device 104 based on e.g., the type of device to which each component is optimized, the type and/or source of data collected at the user device, etc.

Next, at step 304, the user device receives one or more transformation processes. As noted above, the transformation processes are stored at the user device 104 in one embodiment, such as in association with the particular health monitoring application run thereat. The user selects to run a particular transformation process (such as by selecting to run a particular health monitoring application or feature within a health monitoring application).

In order to run the application or feature, at step 306, the transformation components needed are identified from the process instructions. As noted above, the process instructions may indicate components to be executed, an order for execution thereof, as well as other features or aspects of the execution. For example, the process instructions may indicate a data type and/or source on which each transformation component is to be performed. Specifically, for example, heart rate data may be collected from e.g., a wrist-worn device as well as a chest strap; in such cases, the transformation process instructions may indicate that data from the chest strap is to be utilized in a particular step of the process (i.e., component) unless it cannot be found, and in such case heart rate data from the wrist-worn device is to be used instead.

Per step 308 data is collected, sensed, monitored, and/or otherwise obtained at the user device 104. The data may comprise health parameter data in one exemplary embodiment. It is appreciated that the data may be collected upon the occurrence of a triggering event or may be continuously monitored. In either instance, when the transformation process is initialized, the transformation components are applied to the specified ones of the collected data in the order specified by the process instructions to arrive at transformed data at step 310. The transformed data may then be provided to the user at the user device 104 (step 312).

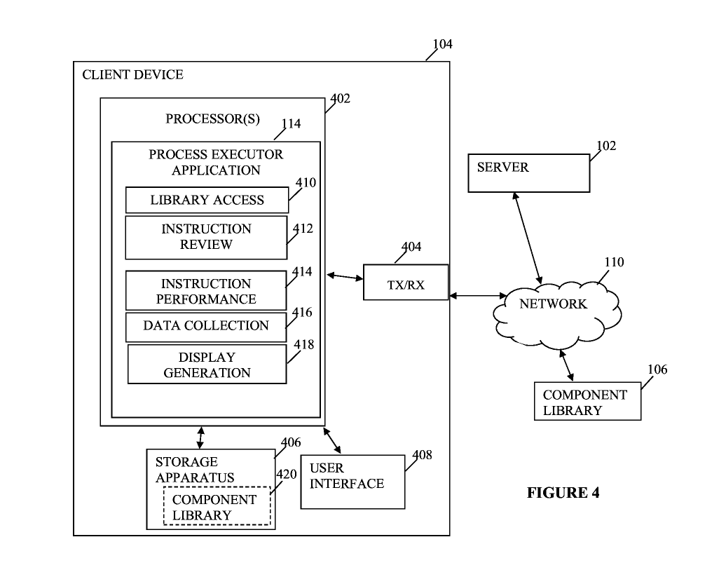

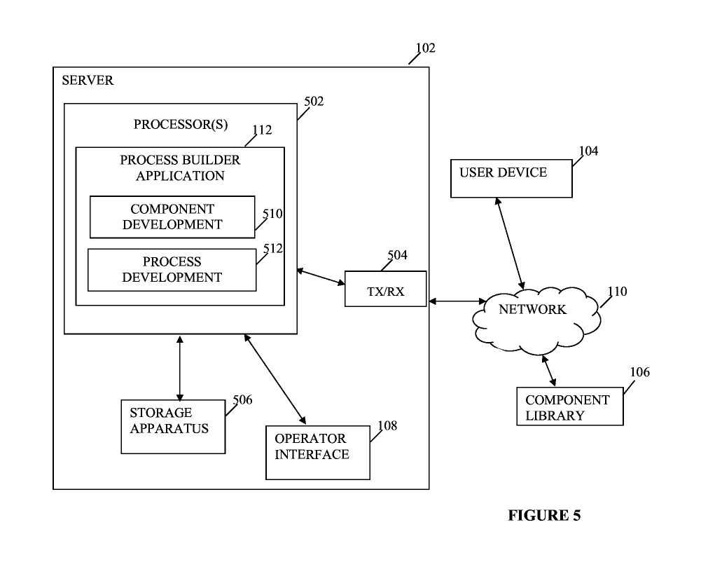

It is noted that the method 300 of FIG. 3 may be performed at a client or user device 104; whereas the method 200 of FIG. 2 may be performed at a server apparatus 102. Exemplary apparatus including an exemplary client device 104 and an exemplary server 102 are now discussed with reference to FIGS. 4-5 below.

### Exemplary User Device

Referring now to FIG. 4, an exemplary user device 104 is provided. The user device 104 may comprise a portable computerized device in one particular embodiment. As illustrated, the device 104 comprises a processor 402, a transceiver 404, a storage device 406, and a user interface 408. As discussed in further detail below, the processor 402 is operable to run at least a process executor application 114 thereon.

As noted above, the user device 104 may further comprise health-monitoring functionality. For example, the user device 104 may comprise a smart phone, smart watch, or other portable electronic device that is configured to both monitor user activity (such as via one or more sensors and/or inputs; not shown). Further, the user device 104 may be configured to access or run a computer application configured to provide data relating to the activity to the user e.g., a health monitoring application. The health monitoring application comprise the process executor application 114 in at least one embodiment.

The transceiver 404 of the exemplary user device 104 illustrated in FIG. 4 enables receipt and transmission of communications to and from the user device 104. For example, the transceiver 404 facilitates the transmission of requests to access particular components from the user device 104 to the component database 106; the transceiver 404 is also configured to receive sensed/measured/obtained raw data from various sensors at the monitoring devices. In addition, the transceiver 404 facilitates transmission of transformation processes and/or components for execution to the device 104 from the server 102 and/or component library 106. As shown, communication is therefore enabled between the user device 104 and the process builder 112 of the server 102 and the component library 106 as discussed herein.

The transceiver 404 may be any of various devices configured for communication with other electronic devices, including the ability to send communication signals and receive communication signals. The transceiver 404 may include different types of transceivers configured to communicate with different networks and systems. Such transceivers are well known and will be recognized by those of ordinary skill in the art. In some embodiments, the transceiver 404 includes at least one transceiver configured to allow the user device 104 to perform wireless communications with the cell towers of the wireless telephony network, as will be recognized by those of ordinary skill in the art. The wireless telephony network may comprise any of several known or future network types. For example, the wireless telephony network may comprise commonly used cellular phone networks using CDMA, GSM or FDMA communication schemes, as well as various other current or future wireless telecommunications arrangements. In some embodiments, the transceiver 404 includes at least one transceiver configured to allow the user device 104 to communicate with any of various local area networks using Wi-Fi, Bluetooth® or any of various other communications schemes.

The storage apparatus 406 of the exemplary user device 104 in FIG. 4 is configured to store local copies of e.g., collected raw activity data (received from the monitoring devices), a client-side version of the aforementioned computer applications (e.g., a health monitoring application and/or the process executor application 114), and any other locally created or stored data. In another embodiment, one or more transformation processes and/or a component library 420 are stored at the storage apparatus 406.

The processor 402 is configured to execute at least a process executor application 114 thereon. The process executor application 114 may be downloaded via a network interface from a web-based server, or alternatively be pre-installed on the device 104 at purchase. The process executor application 114 comprises a plurality of instructions which are configured to, when executed by the processor 402, enable utilization of a distributable or transportable data transformation topology. In one specific embodiment, the process executor application 114 comprises a plurality of functional applications including: a library access application 410, an instruction review application 412, an instruction performance application 414, a data collection application 416, and a display generation application 418. Each of these will be discussed in turn below.

The library access application 410 comprises a plurality of instructions which are configured to, when executed by the processor 402, enable intra-application and/or intra-device communications to facilitate access to a library of transformation process components. In one embodiment, the library access application 410 may enable access of a remote component library, such as component library 106, via a network 110. In another embodiment, the library access application 410 enables access of a component library 420 stored at the user device 104. In either instance, the library access application 410 enables the client device 104 to call specific ones of the components to be executed in association with the performance of a transformation process (as discussed above).

The instruction review application 412 comprises a plurality of instructions which are configured to, when executed by the processor 402, review the process instructions for a particular transformation process in order to identify the components and the data which are needed to execute the transformation process. The application 412 specifically identifies those components which are needed and communicates to the library access application 410 which in turn calls the components. In one variant, this is accomplished via utilization of unique identifiers associated to each component and set forth in the instructions. Alternatively, the components may be listed by location or address within the component library.

The instruction performance application 414 comprises a plurality of instructions which are configured to, when executed by the processor 402, cause the identified transformation components to be executed in an order specified by the process instructions. Additionally, the performance application 414 ensures that the appropriate data is utilized in each component or step. This may be accomplished via the aforementioned metadata tags applied to the data as it is collected. As noted above, the application 414 may refer to a prioritized list of data types, such that the process looks for a first data type and if it cannot be found, continues down a list of data types ordered by preference by the operator who built the process. The instruction performance application 414 may further be configured to ensure that all instructions relating to any aspect or feature of the given transformation process are executed.

In a further embodiment, the instruction performance application 414 may be configured to identify that a particular data collection device which is more accurate for obtaining a particular type of data has been added to a user's set of data collection devices. Upon determining the more accurate device is available, the performance application 414 may determine to utilize the more accurate data in execution of a process (in accordance with the previously mentioned hierarchal list of preferred devices and/or data types).

The data collection application 416 comprises a plurality of instructions which are configured to, when executed by the processor 402, collect, sense, monitor, and/or otherwise obtain health parameter related data. In one embodiment, the data collection application 416 comprises a health monitoring application, such as e.g., UA Record™, MapMyFitness®, MyFitnessPal®, Endomondo®, etc. each owned by assignee hereof. Other health activity related monitoring applications may additionally be utilized as well. It is appreciated that the data collection application 416 may comprise a series of additional components necessary for the separate function of data collection, including e.g., communication components, sensor components, etc. (not shown).

The display generation application 418 comprises a plurality of instructions which are configured to, when executed by the processor 402, enable the generation of a plurality of user interfaces or displays discussed herein. Specifically, one or more user interfaces may be generated which display the aforementioned collected data, display the transformed data, display one or more additional features or “pages” of the health monitoring application, enable the user to interact with particular “pages”, and so forth.

It is appreciated that the user device 104 may comprise additional applications (not shown) which contribute to the functioning thereof as described herein and/or the foregoing functionality may be distributed across more applications or combined into fewer applications. For example, the aforementioned health monitoring application may be provided via a separate application than the process executor application 114. These and other components of the user device 104 will be clear to a person of ordinary skill in the art given the discussion of the functionality herein.

In one embodiment, the aforementioned processing is performed via coordination of a distributed application having client and network-side components. The network-side component may be run at a network entity and the client-side component run at the user device 104.

The herein-described applications enable data transformation topologies to be transported and/or distributed within a network of devices as discussed throughout the disclosure and include e.g., the process executor application 114, the library access application 410, the instruction review application 412, the instruction performance application 414, the data collection application 416, and the display generation application 418. A permanent copy of the programming instructions for these applications (114, 410, 412, 414, 416, and/or 418) may be placed into permanent storage devices (such as e.g., the storage apparatus 406) during manufacture of the user device 104, or in the field, through e.g., a distribution medium (not shown), such as a compact disc (CD), or from a distribution server (not shown) via the network 110. That is, one or more distribution media having an implementation of the agent program may be employed to distribute the agent and program various computing devices.

The herein described distributable transformation topology applications (114, 410, 412, 414, 416, and/or 418) improve the functioning of the user device 104 by enabling it to call upon individual components of a process from a component library to be executed. Furthermore, devices that are able to build call components individually from a component library as disclosed herein can operate to more effectively perform a most recent or updated data transformation processes.

### Exemplary Sever

Referring now to FIG. 5, an exemplary server device 102 is provided. The server 102 may comprise a computerized device having a processor 502, a transceiver 504, a storage device 506, and an operator interface 108. As discussed in further detail below, the processor 402 is operable to run at least a process builder application 108 thereon.

The transceiver 504 of the exemplary server 102 illustrated in FIG. 5 enables receipt and transmission of communications to and from the server 102. For example, the transceiver 504 facilitates the transmission of newly developed and/or updated components, newly developed and/or updated processes to e.g., the user devices 104 and/or the component library 106. The transceiver 504 may also facilitate communications to the server 102 such as from the user devices 104, component library 106 and/or other network components 110 (not shown).

The storage apparatus 506 of the exemplary server 102 in FIG. 5 is configured to store local copies of e.g., the applications run at the processor 502, created transformation processes, process instructions, transformation components, and/or any other locally created or stored data.

The processor 502 is configured to execute at least a process builder application 112 thereon. The process builder application 112 comprises a plurality of instructions which are configured to, when executed by the processor 502, facilitate development of transformation topologies which are able to be transported and/or distributed within a network of devices and enable a network operator or administrator to develop a library of application components which are accessible to a plurality of user devices, and subsequently define a sequence of performance of ones of the components to also develop one or more transformation processes. In one specific embodiment, the process builder application 112 comprises a plurality of functional applications including: a component development application 510, and a process development application 512. Each of these will be discussed in turn below.

The component development application 510 comprises a plurality of instructions which are configured to, when executed by the processor 502, enable a network operator or administrator to develop a plurality of transformation components. The transformation components comprise the individual steps which may be applied to complete a transformation process. In one embodiment, the components are generated via user (or operator) inputs into an operator interface which specify one or more data types (or data collection devices from which data should be obtained), as well as one or more mathematical functions which are to be applied thereto. The operator may manually enter for each transformation process component, an identification of the type and/or source of data to be used therein via utilization of metadata tags, or other unique identifier. The operator may further identify via the component development application 510 the type of device (or devices) for which the component is optimized. Additionally, the application 510 may enable the operator to assign a unique identifier or unique location within a component library for each component; the identifier or address may be used to call the component (discussed elsewhere herein).

The component development application 510 may further enable an operator to access existing components and update one or more aspects thereof. For example, an updated mathematical algorithm may be provided for an existing application to convert heart rate data into e.g., a so-called “intensity” score.

As noted above, as each new transformation component is created, it is provided from the component development application 510 to the individual user devices 104 and/or to the external component libraries 106 via the transceiver 504. Similarly, as changes are made to existing components, these changes are populated to the component storage entities (e.g., at the user device 104 and/or at the external component libraries 106).

The process development application 512 comprises a plurality of instructions which are configured to, when executed by the processor 502, enable a network operator or administrator to develop a plurality of transformation processes from the developed components. As noted above, each transformation process comprises a series of components. In one embodiment, the operator selects the individual components to be applied, and places these within a process instruction. The process instruction may identify the components by e.g., a unique identifier thereof and/or a location thereof within the component library. The process development application 512 may further enable the operator to identify the data type(s) to be utilized and/or a health monitoring device from which the data should be collected. The application 512 enables the operator to place any other aspects or functions into the process instructions as needed to build the transformation process. Updates to existing processes may be similarly created via the process development application 512. As noted above, as each new transformation process is created and/or updated, it is provided from the process development application 512 to the individual user devices 104 for execution thereat (e.g., to the process executor applications 114 of the user devices 104).

It is appreciated that the server 102 may comprise additional applications (now shown) which contribute to the functioning thereof as described herein and/or the foregoing functionality may be distributed across more applications or combined into fewer applications. Including e.g., placement of the component development application 510 and/or the process development application 512 at an individual administrator or operator device (not shown). These and other components of the server 102 will be clear to a person of ordinary skill in the art given the discussion of the functionality herein.

The herein-described applications enable data transformation topologies to be transported and/or distributed within a network of devices as discussed throughout the disclosure and include e.g., the process builder application 112, the component development application 510, and the process development application 512. A permanent copy of the programming instructions for these applications (112, 510, and/or 512) may be placed into permanent storage devices (such as e.g., the storage apparatus 506) during manufacture of the server 102, or in the field, through e.g., a distribution medium (not shown), such as a compact disc (CD), or from a distribution server (not shown) via the network 110. That is, one or more distribution media having an implementation of the agent program may be employed to distribute the agent and program various computing devices.

The herein-described applications (112, 510, and/or 512) improve the functioning of the server 102 by enabling it to create a library of application components which are accessible to a plurality of user devices, and subsequently defining a sequence of performance of ones of the components to complete a transformation process on collected data.

It will be appreciated that variants of the above-described and other features and functions, or alternatives thereof, may be desirably combined into many other different systems, applications or methods. Various presently unforeseen or unanticipated alternatives, modifications, variations or improvements may be subsequently made by those skilled in the art that are also intended to be encompassed by the following claims.

It will be appreciated that the various ones of the foregoing aspects of the present disclosure, or any parts or functions thereof, may be implemented using hardware, software, firmware, tangible, and non-transitory computer readable or computer usable storage media having instructions stored thereon, or a combination thereof, and may be implemented in one or more computer systems.

It will be apparent to those skilled in the art that various modifications and variations can be made in the disclosed embodiments of the disclosed device and associated methods without departing from the spirit or scope of the disclosure. Thus, it is intended that the present disclosure covers the modifications and variations of the embodiments disclosed above provided that the modifications and variations come within the scope of any claims and their equivalents.

What is claimed is:

1. A system configured to enable implementation of data transformation topologies across a network of devices, said system comprising:
at least one network apparatus comprising at least one first computer program, said first computer program comprising a plurality of instructions which are configured to, when executed, cause said network apparatus to:
generate a plurality of components, each of said plurality of components comprising at least one mathematical process to be applied; and
create an instruction set, said instruction set comprising an ordered list of multiple ones of said plurality of components, and information configured to identify one or more specific data types to be utilized with each of said multiple ones of said plurality of components; and
enable a plurality of user devices to:
receive access to said plurality of components;
receive said instruction set;
obtain said identified one or more of said specific data types; and
perform said multiple ones of said plurality of components in said order provided by said instruction set to arrive at transformed data.

2. The system of claim 1, wherein said plurality of instructions of said first computer program are further configured to, when executed, cause said network apparatus to:
access at least one of said plurality of components;
alter at least one aspect of said at least one mathematical process to be applied via said accessed at least one of said plurality of components; and
enable said plurality of user devices to access said updated at least one of said plurality of components.

3. The system of claim 1, wherein said plurality of instructions of said second computer program are further configured to, when executed, cause said user devices to:
access said instruction set;
alter at least one aspect of said ordered list and/or said information configured to identify one or more specific data types to be utilized; and
enable said plurality of user devices to access said updated instruction set.

4. The system of claim 1, wherein:
said one or more of said specific data types comprise health-parameter data and said user device is configured to obtain said health parameter data from at least one sensor apparatus; and
said transformed data is provided to said user in association with a health-monitoring application run at one of said plurality of user devices and displayed via a display device associated therewith.

5. The system of claim 1, wherein said identification of said one or more of said specific data types comprises an identification based on a metadata tag associated with said data indicating at least a device from which said data was sensed.

6. The system of claim 1, wherein each of said plurality of components further comprises at least a unique identifier associated thereto and wherein said ordered list of said instruction set comprises a list of said unique identifiers of said multiple ones of said plurality of components.

7. A non-transitory, computer readable medium comprising a plurality of instructions which are configured to, when executed, cause a user device to:
receive access to a library comprising a plurality of processing functions;
receive a transformation process instruction, said instruction comprising individual ones of said plurality of processing functions to be executed and a pre-defined order in which said individual ones of said plurality of processing functions are to be executed;
obtain a plurality of data;
identify at least a portion of said data for use in said transformation process;
access said individual ones of said plurality of processing functions identified in said instruction;
execute said individual ones of said plurality of processing functions on said at least portion of said data in said pre-defined order to arrive at transformed data; and
provide said transformed data to a user.

8. The computer readable medium of claim 7, wherein each of said processing functions comprises a mathematical process to be applied to said identified at least portion of said data.

9. The computer readable medium of claim 7, wherein said plurality of data comprises health-parameter data and said user device is configured to obtain said health parameter data from at least one sensor apparatus.

10. The computer readable medium of claim 9, wherein said transformed data is provided to said user in association with a health-monitoring application run at said user device and displayed via a display device associated to said user device.

11. The computer readable medium of claim 7, wherein said library comprises at least one of a storage apparatus associated to said user device, or a remote apparatus configured to store said plurality of processing functions and in communication with said user device.

12. The computer readable medium of claim 7, wherein said identification of said at least portion of said data for use in said transformation process comprises an identification based on a metadata tag associated with said data indicating at least a device from which said data was sensed.

13. A network apparatus configured to enable transformation topologies to be distributable to a plurality of user devices, said apparatus comprising:
one or more interfaces;
a storage apparatus; and
a processor configured to execute at least one computer application thereon, said computer application comprising a plurality of instructions which are configured to, when executed, cause said network apparatus to:
generate a plurality of components, each of said plurality of components comprising at least one mathematical process to be applied;
transmit said plurality of components to at least one storage apparatus associated with at least one of said plurality of user devices;
create an instruction set, said instruction set comprising an ordered list of multiple ones of said plurality of components, and information configured to identify one or more specific data types to be utilized with each of said multiple ones of said plurality of components; and
provide said instruction set to said at least one of said plurality of user devices for execution thereby.

14. The network apparatus of claim 13, wherein each of said components further comprises at least a unique identifier associated thereto and wherein said ordered list of said instruction set comprises a list of said unique identifiers of said multiple ones of said plurality of components.

15. The network apparatus of claim 13, wherein said execution of said instruction set by said at least one of said plurality of user devices comprises said user device calling each of said multiple ones of said plurality of components from said storage apparatus.

16. The network apparatus of claim 15, wherein said execution of said instruction set by said at least one of said plurality of user devices further comprises said user device identifying said specific data types to be utilized based at least in part on metadata tags associated with collected data at said user device.

17. The network apparatus of claim 13, wherein said plurality of instructions are configured to, when executed, cause said network apparatus to:
access at least one of said plurality of components;
alter at least one aspect of said at least one mathematical process to be applied via said accessed at least one of said plurality of components; and
provide said updated at least one of said plurality of components to said at least one storage apparatus associated with at least one of said plurality of user devices.

18. The network apparatus of claim 17, wherein said alteration to said at least one aspect comprises an alternation of an identity of a source of said specific data type to be used therewith.

19. The network apparatus of claim 18, wherein said alteration is based at least in part on a determination that a more accurate source of said specific data type is available.
